(19) United States
(12) Patent Application Publication
(10) Pub. No.: US 2025/0275928 A1
(43) Pub. Date: Sep. 4, 2025

(54) NANOPARTICLE-BASED ABUSE-DETERRENT FORMULATIONS AND METHODS OF MAKING AND USING

(71) Applicant: Purdue Research Foundation, West Lafayette, IN (US)

(72) Inventors: Yoon Yeo, West Lafayette, IN (US); Sheryhan F. Gad, Lawrence, KS (US)

(73) Assignee: Purdue Research Foundation, West Lafayette, IN (US)

(21) Appl. No.: 18/702,531

(22) PCT Filed: Oct. 17, 2022
(86) PCT No.: PCT/US2022/046876

§ 371 Date: Apr. 18, 2024

Related U.S. Application Data
(60) Provisional application No. 63/256,746, filed on Oct. 18, 2021.

(51) Int. Cl. A61K 9/51; A61K 9/20; A61K 31/485

(52) U.S. Cl. CPC A61K 9/5146; A61K 9/2013; A61K 9/2054; A61K 9/2081; A61K 9/2095; A61K 9/5115; A61K 9/5192; A61K 31/485

(57) ABSTRACT

An abuse-deterrent formulation comprising an abuse-prone drug encapsulated in nanoparticles, a gel-forming polymer, and pharmaceutically acceptable excipients; method of use; and method of making.

### CROSS-REFERENCE TO RELATED APPLICATIONS

This application claims priority to U.S. Provisional Patent application No. 63/256,746, which was filed Oct. 18, 2021, which is incorporated herein by reference in its entirety.

### GOVERNMENT RIGHTS

This invention was made with government support under DA048074 awarded by the National Institutes of Health. The government has certain rights in the invention.

### TECHNICAL FIELD

The present disclosure generally relates to an abuse-deterrent formulation and particularly to a nanoparticle-based abuse-deterrent formulation in which a drug prone to abuse is encapsulated, a method of making, and a method of using.

### BACKGROUND

This section introduces aspects that may help facilitate a better understanding of the disclosure. Accordingly, these statements are to be read in this light and are not to be understood as admissions about what is or is not prior art.

Driven by increasing opioid addiction, drug overdose is a leading cause of death in the United States. New reports showed that deaths related to the overdose of synthetic opioids went up in 2020, with the disruption of daily life, isolation, and anxiety due to the pandemic. Opioids are potent analgesics if taken as prescribed. However, drug addicts alter the method of ingestion to feel the effects as quickly as possible. The addicts pulverize pills to snort, smoke, or prepare to inject. The drug is readily extracted in powder form by common household solvents such as ethanol, vinegar, soda, etc. These alternative strategies result in a rapid and dangerous increase in the drug concentration in the blood, often to the fatal level.

Pharmaceutical efforts have focused on maintaining the balance between the benefits and the risks of opioid therapy, utilizing various abuse-deterrent formulations (ADFs). ADFs refer to special dosage forms designed to discourage physical and chemical tampering with the formulation without losing the therapeutic effects of opioids. Most ADFs rely on carrier polymers, which have thermal, gelling, swelling, mechanical, binding, or film-forming properties, thereby limiting the diffusion of the drug out of the pill under different extraction conditions. For example, ADFs utilize polymers, such as high molecular weight polyethylene oxide (PEO), that form a viscous gel when contacted with water, deterring both intravenous injection and extraction. Other formulations make pills more difficult to crush. Alternatively, agonist/antagonist combinations or aversive agents are included in ADFs to act as pharmacological deterrents that alter the pharmacological effect of the opioid if manipulated by abusers.

Several ADFs have proven effective in decreasing the rate of substance abuse by 75%. However, abusers have also developed more complex extraction strategies to circumvent the ADF technologies. When manipulated in water, ADF polymers are mostly used at enough concentration to impart viscosity or swelling. However, the gelling property can be lost entirely or decreased if the polymer is heated with a candle, lighter, or microwave, giving a chance to the abusers to extract the drug. Moreover, polymers do not prevent drug extraction by organic solvents, such as ethanol or acetone. Addicts can put the drug that contains an entire 12-hour dose in vodka and then drink the vodka/opioid solution at once. They can also extract the drug using a volatile organic solvent, evaporate the solvent after filtering the undissolved polymer, and dissolve the drug in water to inject. Gelling properties of some polymers can also be affected by the pH and salts in the solution. Many of these techniques are posted on the internet and made available to the public without restrictions.

Therefore, there is a strong need for new technology to complement the limitations of current ADFs. It is an object of the present disclosure to meet such a need. This and other objects and advantages, as well as inventive features, will be apparent from the detailed description provided herein.

### SUMMARY

The present disclosure is related to a nanoparticle-based abuse-deterrent formulation which can deter abuse of a drug by preventing aqueous or organic solvent extraction, injection, and crisping of the drug but provide necessary analgesia if taken as prescribed.

Provided is an abuse-deterrent formulation (ADF) comprising (i) a drug encapsulated in nanoparticles (NPs), wherein the drug is prone to abuse and the nanoparticles are stabilized with polytannic acid alone or in combination with iron, wherein the nanoparticles comprise poloxamer-based surfactants, (ii) a gel-forming polymer, and (iii) one or more pharmaceutically acceptable excipients.

In exemplary embodiments, the poloxamer-based surfactant is Pluronic. NPs comprising Pluronic can be stabilized with polytannic acid alone or in combination with iron.

In this ADF, the drug is encapsulated in stabilized NPs which are resistant to extraction by organic solvents that can be used for drug abuse. The organic solvents that can be used for drug extraction are common household solvents. Examples of household solvents include, but are not limited to, water, ethanol, acetone, an alcoholic-containing beverage, vinegar, an electrolyte solution, a sodium bicarbonate solution, or a carbonated beverage comprising phosphoric acid alone or in further combination with caffeine.

NPs can have a diameter range from between about 300 nm to about 800 nm, preferably from between about 300 nm to about 500 nm.

In exemplary embodiments, gel-forming polymer are selected from a starch-based polymer such as sodium starch glycolate (SSG), a polyacrylate such as sodium polyacrylate starch (SPS), a Carbopol® (CP), a polyethylene oxide (PEO) such as Polyox™ 7 MDa, a cellulose derivative such as hydroxyethyl cellulose (HEC), a polysaccharide such as xanthan gum (XG), chitosan (CS), and a combination of two or more polymers.

The gel-forming polymers are resistant to common household aqueous solvents through their gelation property. Examples of aqueous solvents include, but are not limited to, water, a sodium bicarbonate solution, a sodium chloride solution, a 5% acetic acid solution, 0.1 M hydrochloric acid solution, or a 0.1 M sodium hydroxide solution.

Example of a drug that can be abused is selected from the group consisting of an opioid, a sedative, a central nervous system (CNS) depressant, a CNS stimulant, and a hallucinogen. In exemplary embodiments, the drug is an opioid.

The ADF can be administered orally in the form of a tablet. Examples of the pharmaceutically acceptable excipient used in ADF include, but are not limited to, Avicel and magnesium stearate. The gel-forming polymer can be used in a tablet in the amount of 60 wt % of 1 g of a tablet. The drug can be encapsulated in an amount of 10 wt % of NPs.

A method of deterring abuse of a drug, which method comprises providing an abuse-deterrent formulation comprising (i) a drug encapsulated in NPs, wherein the drug is prone to abuse and the NPs are stabilized with polytannic acid alone or in combination with iron, wherein the nanoparticles comprise poloxamer-based surfactants, (ii) a gel forming polymer, and (iii) one or more pharmaceutically acceptable excipients, wherein the formulation deters abuse of the drug by preventing an aqueous or organic solvent extraction, injection, and crisping of the formulation.

A process for the preparation of an abuse-deterrent formulation, which process comprises:
a) incorporating an abuse-prone drug into NPs;
b) stabilizing NPs using polytannic acid alone or in combination with iron; and
c) co-formulating the NPs with a gel-forming polymer and one or more pharmaceutically acceptable excipients.

The drug-loaded NPs can be lyophilized without compromising their integrity and functions. They have a low systemic exposure and are preferentially accumulated in the liver and degraded therein without causing-abusive damage to the patient if injected.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIG. 1 shows the gelation screening of different polymers or their combinations by inversion in DI water (H₂O), 0.1 M hydrochloric acid (HCl), 5% acetic acid, sodium chloride (NaCl), sodium bicarbonate (NaHCO₃), and sodium hydroxide (NaOH). A scoring scale of 0-5 represents:
0: unable to gel in 1 h; 1: gel in 30-60 min; 2: gel in 10-30 min; 3: gel in 5-10 min; 4: gel in 1-5 min; and 5: forms a gel within 1 min. The screened polymers are SSG: sodium starch glycolate; SPS: sodium polyacrylate starch; CP: Carbopol®; PEO: Polyox™ 7 MDa; HEC: hydroxyethyl cellulose; XG: xanthan gum; and CS: chitosan.

FIG. 2 shows the preparation of polytannic acid/iron-stabilized Pluronic F127 nanaoparticles (NPs) by a quasi-emulsion method (QpTA NPs).

FIG. 3a shows transmission electron microscopy (TEM) images of QpTA NPs without (top) and with thebaine (bottom).

FIG. 3b shows the size distribution of QpTA NPs measured by dynamic light scattering (Zetasizer nano-ZS90), n=6 independent experiments, 3 runs each.

FIG. 3c shows the Z-average and polydispersity index (PDI) of fresh or freeze-dried QpTA NPs. Numbers indicate the ratio of trehalose to NP, n=2 independent experiments.

FIG. 4a shows various household solvents used by drug abusers.

FIG. 4b shows % Thebaine (THB) extracted by common household solvents as a free drug, fresh QpTA NPs, and freeze-dried QpTA NPs (1:20 NP: trehalose).

FIG. 5a shows the % THB extracted by 100% ethanol for 1 hour as fresh QpTANPs or freeze-dried Avicel/NPs mixed with polymers. n=2 independent experiments. XG, HEC, and CS numbers indicate the concentration of XG, HEC, and CS used during the experiment in % w/v.

FIG. 5b shows the % THB extracted by ethanol for 1 hour from QpTA NPs co-dried with trehalose (Tre) via freeze-drying (FD) and Avicel (Av) via FD or vacuum-drying (VD).

FIG. 6a shows the % THB extracted by 100% ethanol for 1 hour as freeze-dried Avicel/QpTA NPs mixed with XG+CS with different concentrations of Magnesium stearate (0.25% and 0.75%).

FIG. 6b shows the % THB extracted by 100% ethanol for 1 hour as freeze-dried Avicel/QpTA NPs mixed with XG+CS with different compression forces (2500 Ib and 3500 Ib) using 0.25% Mg stearate and crushed before the extraction.

FIG. 7a shows the % THB extracted after manipulating from free drug tablets ADF-NP (F-tab). The experiment was done on 3 independent batches, and data were presented as mean±SD.

FIG. 7b shows the % THB extracted after manipulating ADF+NP (NP-tab) with absolute ethanol and acetone. The experiment was done on 3 independent batches, and data were presented as mean±SD.

FIG. 8 shows the % drug recovered after crisping of physical mixtures of polymer (20 mg XG+60 mg CS) with unformulated drug (free THB), Blank QpTA+THB, or THB loaded QpTA at 180° C. for 10 min.

FIG. 9 shows the % drug recovered after crisping crushed tablets of the unformulated drug (F-Tab) or THB-loaded QpTA NPs (NP-tab) at 180° C. for 13 min.

FIG. 10a shows the dissolution of NP-Tab in comparison to F-Tab in 25 mL of SGF, wherein cumulative % release in SGF within 8 hour.

FIG. 10b shows the % THB remained after the complete dissolution of NP-Tab and F-Tab compared to drug only, indicating instability of THB in SGF after 8 hour.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, 99%, 99.5%, 99.9%, 99.99%, or at least about 99.999% or more of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “abuse-deterrent formulation” is used to refer to a formulation that reduces the potential for improper administration of drugs but that delivers a therapeutically effective dose of the drug when administered as directed. Improper administration includes tampering with the dosage form and/or administering the drug by any route other than instructed.

The term “pharmaceutically acceptable excipients” are substances other than the active pharmaceutical ingredient (API) that have been appropriately evaluated for safety and are intentionally included in a drug delivery system.

The term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable excipients, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art-recognized route of parenteral administration.

Illustrative means of parenteral administration include needle (including microneedle) injectors, needle-free injectors, and infusion techniques, as well as any other means of parenteral administration recognized in the art. Parenteral formulations are typically aqueous solutions which may contain excipients such as salts, carbohydrates, and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of a composition or formulation depends on several factors, including: the administration method, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

The term “therapeutically effective amount” or “effective amount” as used herein refers to that amount of the drug in the abuse-deterrent formulation.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of drug in the abuse-deterrent formulation described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The present disclosure generally relates to a nanoparticle-based (NP-based) abuse-deterrent formulation. The formulation includes an encapsulation system that deters drug (e.g., opioid) extraction but does not compromise the necessary therapeutic effect (e.g., analgesia) if taken as prescribed.

In view of the above, provided is an abuse-deterrent formulation comprising (i) a drug encapsulated in nanoparticles (NPs), wherein the drug is abuse prone and the nanoparticles are stabilized with polytannic acid alone or in combination with iron, wherein the nanoparticles comprise poloxamer-based surfactants, (ii) a gel-forming polymer, and (iii) one or more pharmaceutically acceptable excipient.

The NPs can be poloxamer-based surfactants. In some embodiments, the surfactants can be poloxamers such as a poloxamer sold using the trademark Pluronic®. The Pluoronic comprise a chain of hydrophobic polypropylene oxide (PPO) and hydrophilic polyethylene oxide (PEO) arranged in a PEO-PPO-PEO manner to form a triblock copolymer. The example of surfactants includes, but is not limited to, Pluronic F127. The NPs comprising poloxamer-based surfactants can be stabilized with polytannic acid alone or in combination with iron.

In exemplary embodiments, NPs comprising a drug and poloxamer-based surfactants such as Pluronic are stabilized with polytannic acid alone or in combination with iron. The stabilized NPs are resistant to extraction by organic solvents that can be used for drug abuse. The abuse of the drug can be deterred by encapsulating them in NPs with polytannic acid, alone or in further combination with iron, which renders the drug-resistant to extraction by common household solvents. The stabilized NPs also prevent the crisping of the drug. Polytannic acid and iron accelerate the thermal destruction of the drug and, thus, discourage the drug's abuse by crisping.

The abuse of the drug can be deterred by encapsulating it in the NPs, which are resistant to an organic solvent that can be used for drug abuse, particularly to the organic solvent that can be used for extraction of the drug. Examples of organic solvents that can be used for drug extraction are common household solvents.

In exemplary embodiments, the common household solvents are selected from water, alcohols such as ethanol, acetone, an alcoholic-containing beverage, vinegar, an electrolyte solution, a sodium bicarbonate solution, or a carbonated beverage comprising phosphoric acid alone or in further combination with caffeine. Examples of the carbonated beverage comprising phosphoric acid include, but are not limited to, cola products such as the products sold under the trade name Coca-Cola®, Coke®, Pepsi®.

Examples of the drug that can be abused include, but are not limited to, opioids, sedatives, central nervous system (CNS) depressants, CNS stimulants, or hallucinogens. In exemplary embodiments, the drug is an opioid, such as Thebaine (THB).

NPs can be used in combination with gel-forming polymers. Gel-forming polymers can act as a physical barrier to deter drug abuse through their gelation properties in an aqueous solution, whereas NPs serve as a chemical barrier against solvent extraction. A combination of NPs and gel-forming polymers can protect the drug from solvents such as ethanol and acid. NPs are resistant to organic solvents, whereas gel-forming polymers resist aqueous solvents through gelation or swelling properties.

Gel-forming polymers are selected based on their ability to form a gel in aqueous common household solvents with different pH values. Examples of gel-forming polymers include, but are not limited to, starch-based polymers such as sodium starch glycolate (SSG), polyacrylates such as sodium polyacrylate starch (SPS), Carbopols® (CP), polyethylene oxides (PEO) such as Polyox™ 7 MDa, cellulose derivatives such as hydroxyethyl cellulose (HEC), polysaccharide such as xanthan gum (XG), chitosan (CS) and combination of two or more polymers. These gel-forming polymers do not form gels in organic solvents.

Examples of the common aqueous solvents to which gel-forming polymers are resistant include, but are not limited to, water, NaHCO₃ solution, NaCl solution, 5% acetic acid solution, 0.1M HCl solution, and 0.1 M NaOH solution.

In exemplary embodiments, a process for the preparation of an abuse-deterrent formulation is provided, which process comprises:
a. incorporating an abuse-prone drug in NPs;
b. stabilizing NPs using polytannic acid alone or in combination with iron; and
c. co-formulating NPs with a gel-forming polymer and one or more pharmaceutically acceptable excipients.

The process involves building an encapsulation system that deters opioid extraction. The drug-loaded NPs can be formulated by any suitable method known in the art, such as the quasi-emulsion method. In the quasi-emulsion method, the drug is mixed with Pluronic F127, in the presence of a solvent. The aqueous phase containing the drug and Pluronic F127 creates a transient water-in-oil emulsion based on the viscosity of glycerol affording drug-loaded NPs. The drug-loaded NPs in the aqueous phase are stabilized using polytannic acid alone or in combination with iron. The drug-loaded Quasi polytannic acid (QpTA) NPs are co-formulated with a gel-forming polymer and one or more pharmaceutically acceptable excipients to obtain the abuse-deterrent formulation. QpTA NPs are spherical capsules. The size, morphology, and encapsulation efficiency of NPs were characterized by dynamic light scattering (Zetasizer Nano-ZS90), transmission electron microscopy, and reverse phase (RP) high-performance liquid chromatography, respectively.

In exemplary embodiments, the NPs are Pluronic NPs stabilized by polytannic acid alone or in combination with iron. The gel-forming polymer is selected from starch-based polymers such as SSG, polyacrylates such as SPS, CPs, PEOs such as Polyox™ 7 MDa, cellulose derivatives such as HEC, polysaccharide such as XG, CS and a combination of two or more polymers.

Any suitable solvent as known in the art can be used in the quasi-emulsion method. Examples of suitable solvents include, but are not limited to, glycerol, and water.

QpTA NPs with or without THB are spherical. The diameter of NPs can range between about 300 nm to about 800 nm (such as about 300 nm to 800 nm or 300 nm to about 800 nm), preferably between 300 nm to about 500 nm (such as about 300 nm to 500 nm or 300 nm to about 500 nm). QpTA NPs have a drug loading capacity of ≤10 wt %.

The drug-loaded NPs have a low systemic exposure and are preferentially accumulated in the liver and degraded therein without causing-abusive damage to the patient if injected. The NPs are too small to crush and hence can minimize the effect of physical damage to ADF by mechanical forces. If injected, NPs will be preferentially accumulated in the liver, where the drugs are metabolized into inactive forms.

The drug-loaded NPs can be lyophilized without compromising their integrity. QpTA NPs can be freeze-dried with trehalose, for example, at different ratios. Trehalose is an example of an additive that is used to prevent the aggregation of nanoparticles during the lyophilization process. Freeze-dried NPs, comprising NPs and trehalose in a ratio of about 1:20 can have the same average diameter as fresh NPs, indicating that QpTA NPs can retain the original size after being freeze-dried and redispersed.

The chemical extractability of THB in the gel-forming polymers with or without excipients was tested in absolute ethanol. The polymers did not affect the extractability of THB by ethanol. Thus, the gel-forming polymer alone is not adequate to deter drug abuse by its extraction in the organic solvent.

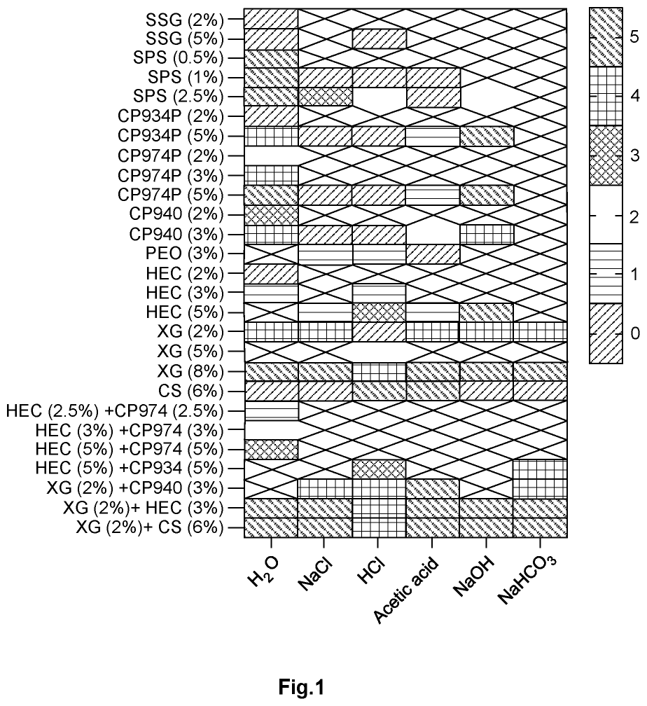

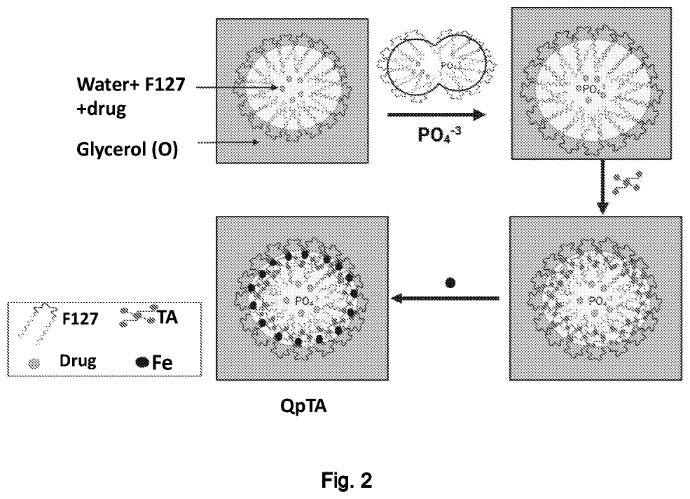

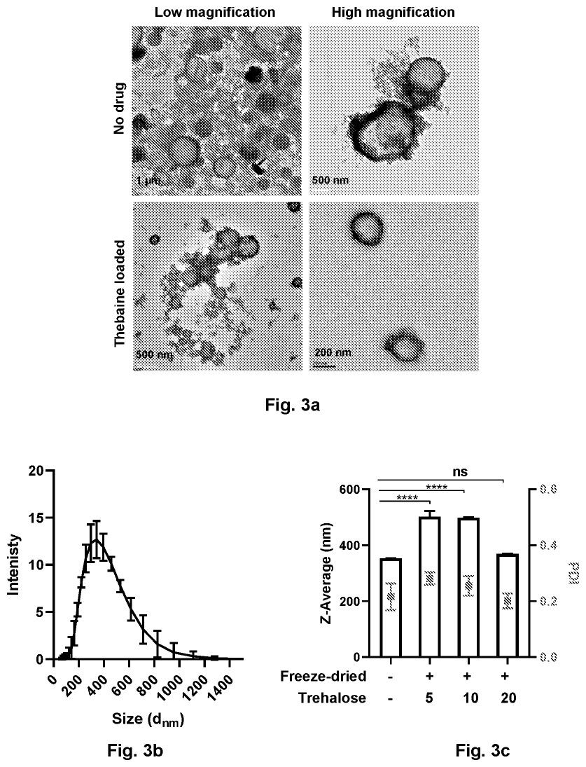

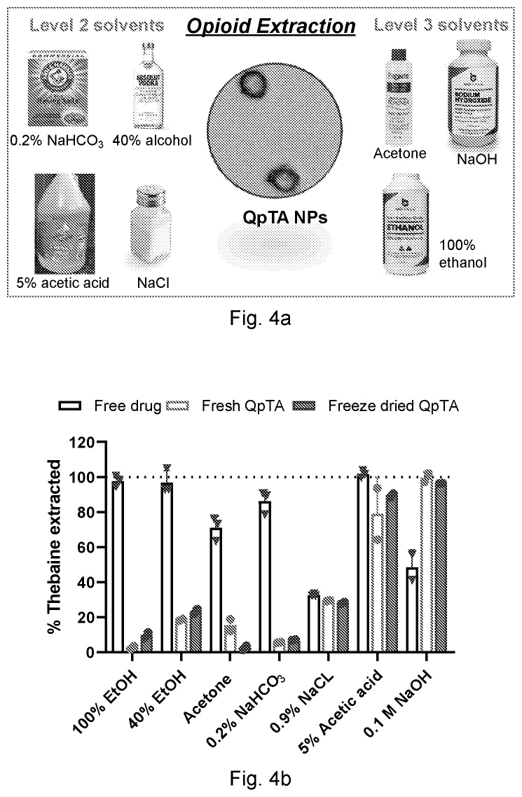

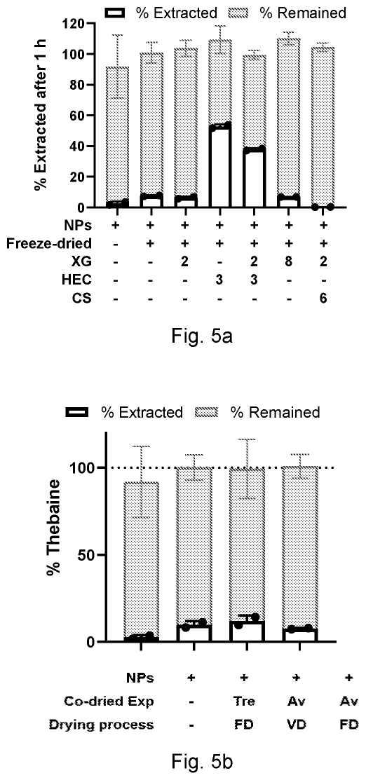

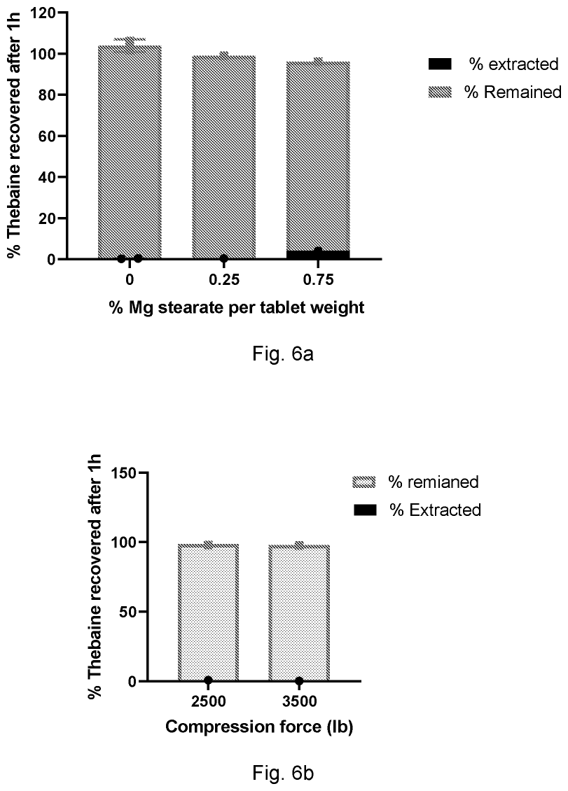

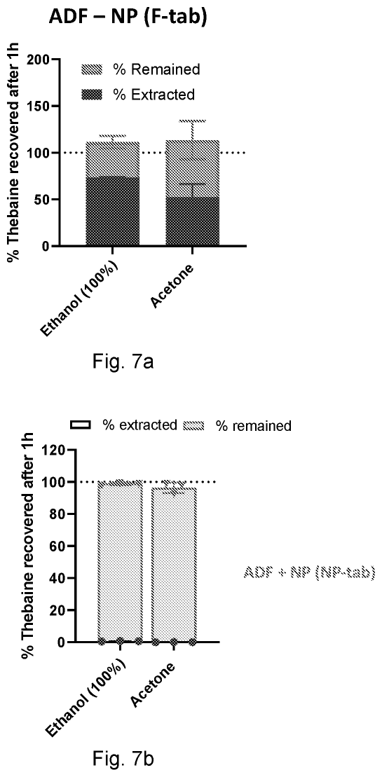

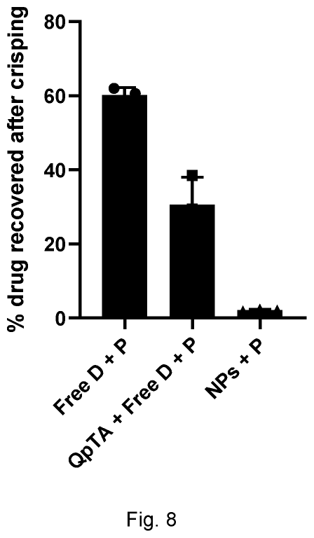

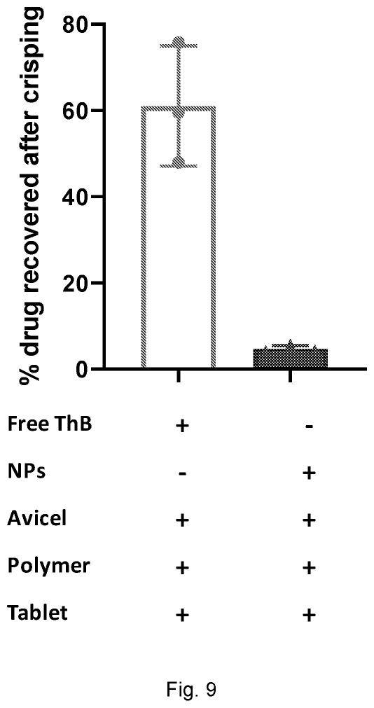

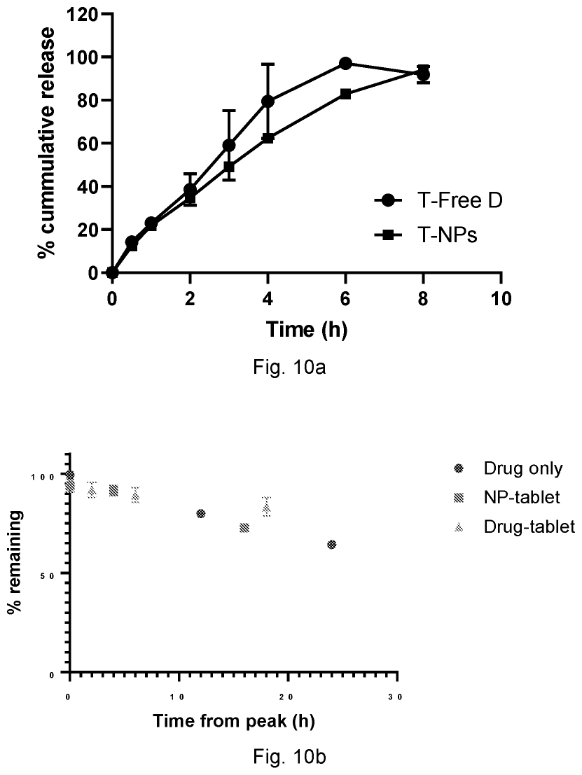

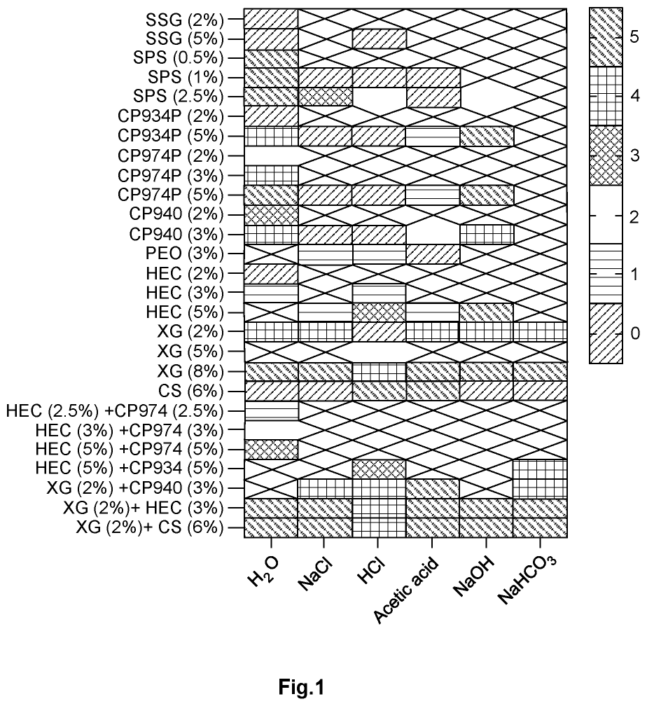

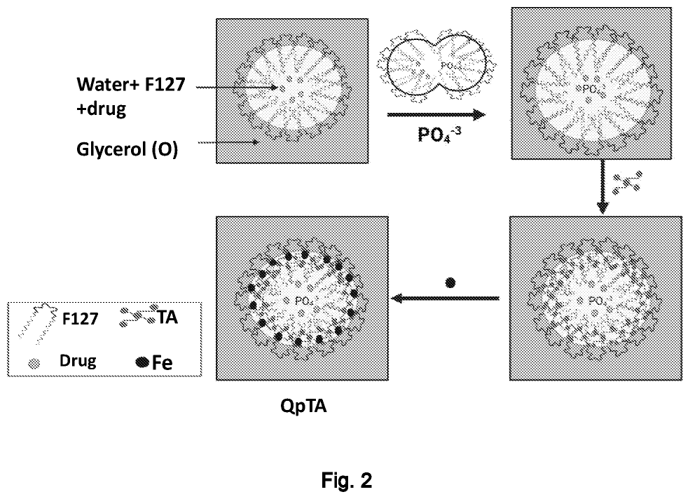

THB was encapsulated in NPs to study chemical extractability. THB was encapsulated in polytannic acid/iron-stabilized Pluronic F127 NPs by a quasi-emulsion method (QpTA NPs) as shown in FIG. 2. to afford THB-loaded NPs.

The chemical extractability of THB encapsulated in QpTA NPs was studied using common household solvents. The results of the studies demonstrated that QpTA NPs prevented the extraction of the THB in most common household solvents such as absolute ethanol, 40% ethanol, acetone, and 0.2% NaHCO₃, unlike unformulated THB, which dissolves completely in most solvents at equivalent concentrations. However, NPs did not prevent extraction by 5% acetic acid (vinegar) or 0.1 M NaOH, allowing ˜100% of the drug to be extracted in 1 hour. There was no significant difference in extractability between fresh and freeze-dried drug-loaded QpTA NPs. Thus, the freeze-drying process does not affect size and stability of the NPs.

The compatibility of gel-forming polymer with QpTAs was studied. XG and CS were most compatible with QpTAs, indicating that XG and CS and their combination can protect the dried NPs from ethanol extraction.

QpTAs can provide an additional barrier to resist the common household solvents and complement the polymer-based ADF strategies. Thus, ADF formulation comprising drug-loaded NPs with a gel-forming polymer prevents drug extraction by a household solvent/medium.

A method of deterring abuse of a drug, which method comprises providing an abuse-deterrent formulation comprising (i) a drug encapsulated in NPs, wherein the drug is prone to abuse and the NPs are stabilized with polytannic acid alone or in combination with iron, wherein the NPs comprise poloxamer-based surfactants, (ii) a gel-forming polymer, and (iii) one or more pharmaceutically acceptable excipients, wherein the formulation deters abuse of the drug by preventing an aqueous or organic solvent extraction, injection, and crisping of the formulation. In exemplary embodiments, the abuse-deterrent formulation is in the form of a tablet.

The tablets can be formulated by combining QpTA NPs with gel-forming polymers using the excipients such as Avicel or magnesium stearate. The formulated nanoparticle tablets (NP-tablets) were tested for gelation, syringeability, and injectability. The NP-tablet comprises at least about 60% wt of gel-forming polymer in 1 gram of tablet. NP-tablet were crushed and mixed with different solvents. The crushed powder formed a gel in <5 minutes in all aqueous solvents, confirming the ability of gel-forming polymers such as XG+CS to deter drug retrieval in aqueous solvents by gelation. The chemical extractability of NP-tablet with organic solvent results confirmed that QpTA NP provides an additional barrier against organic solvent manipulation.

In exemplary embodiments, an amount of the drug encapsulated in the NPs can be about 10 wt % of nanoparticles. An amount of the gel-forming polymer used can be about 60 wt % of 1 gram of the tablet.

The ADF formulation can deter abuse of the drug; nevertheless, it can provide a drug and the desired analgesic effect if it is taken as recommended orally. The formulation can be administered orally. The effective amount of the formulation can be an amount that delivers a dose of the drug sufficient to provide the desired therapeutic effect, such as analgesia, while simultaneously deterring abuse of the drug by resisting extraction of the drug. An exemplary therapeutically effective amount of the ADF can be the amount that provides the necessary analgesia when administered in single or divided doses at the practitioner's discretion.

One more way the abuser can abuse the drug is crisping. Crisping is one of the creative approaches, where abusers use a heat source such as a lighter, oven, or microwave to pre-heat ADF powders until it destroys the ADF polymer turning the color to light brown and thereby its gelling property. Upon crisping, the resulting drug powder does not form a viscous mass upon hydration and is relatively easy to inject. Crisping methods are readily available on websites.

The ADF formulation, NP-Tablet, can deter drug abuse via crisping. NP encapsulation makes the drug withstand heating. The study shows that an insignificant fraction of THB (only 2.1% of total drug) from the polymer and drug-loaded QpTA NP mixture was accessible after crisping, whereas 60.2% was recovered from the polymers and unformulated THB mixture. This result suggests that QpTA NPs can induce almost complete drug destruction, whereas >60% of the drug in an unformulated mixture can tolerate crisping. In other words, abusers may not be satisfied by crisping NP-Tablet.

With various embodiments having been described, it will be apparent to those of ordinary skill in the art that many more embodiments and implementations are possible. Accordingly, the embodiments described herein are examples, not the only possible embodiments and implementations.

### EXPERIMENTAL

### 1. Screening of ADF Polymers

### 1a) Gelation Screening

Starch-based, polyacrylates, cellulose derivatives, and polysaccharides, which are used for existing ADF formulations due to their gelation/swelling properties, were screened for the ability to form a gel in aqueous common household solvents with different pH values. Our selection criteria are polymers that form a gel or viscous solution at a concentration of 6% w/v or lower, irrespective of pH, in 5 min. The 6% w/v criterion was selected based on the assumption that a tablet (1 g) contains at least 600 mg polymeric excipient(s), and an abuser may use no more than 10 mL of solvent for drug extraction from each tablet (>600 mg/<10 mL=>6%). Starch-based polymer such as sodium starch glycolate (Explotab, SSG), sodium polyacrylate starch (SPS, superabsorbent), Carbopol® (CP, polyacrylate highly swellable polymer), Polyox™ 7 MDa (PEO), cellulose derivatives such as hydroxyethyl cellulose (HEC,), polysaccharide such as xanthan gum (XG), chitosan (CS), and combinations of two selected polymers were tested. First, each polymer was put in water and observed whether it formed a gel when inverted. Scoring scale 0-5 was used to represent the time for gelation (see, for example, FIG. 1). score 0 is: unable to gel in 1 h; score 1 is: gel in 30-60 min; score 2 is: gel in 10-30 min; score 3 is: gel in 5-10 min; score 4 is: gel in 1-5 min; and score 5 is: forms a gel in 1 min. The polymer that formed a gel within 5 min or less in water (score 4 or 5) was further tested against other solvents such as 0.1 M NaOH, 0.2% NaHCO₃, 0.9% NaCl, 5% acetic acid, and 0.1 M HCl.

Most of the tested polymers did not form gels in all tested aqueous solvents (FIG. 1). Those that formed gels in water lost their gelling property in acidic solutions such as 5% acetic acid and 0.1 M HCl or ionic solution such as 0.9% NaCl. An opposite case was also observed. HEC at 5% w/v took ˜60 min to gel in water, acetic acid, or sodium chloride (score 1) but formed stable gel in HCl in 60 min at 3% w/v (score 1) or 10 min at 5% w/v (score 3). CS formed a gel within 1 min (score 5) at 6% w/v in 0.1 M HCl and 5% acetic acid but did not form a gel in other solvents. In contrast, XG at 2% w/v formed a gel in 5 min (score 4) in all tested aqueous solvents except for 0.1 M HCl (score 0). XG at 8% w/v formed a gel in 0.1 M HCl within 5 min (score 4) and its gelation time decreased (score 5) in all other tested solvents. Combination with other polymers also enhanced the gelation of XG in aqueous solvents. XG 2% w/v+HEC 3% w/v and XG 2% w/v+CS 6% w/v formed gels instantaneously (FIG. 1) in all tested aqueous solvents, irrespective of pH. However, they did not gel in absolute ethanol, acetone and 40% ethanol. This indicates that XG and its combinations with other polymers may deter drug retrieval in aqueous solvents but do not provide enough protection from organic solvents by gelation.

1b) Extraction of Unformulated Drug from Selected Polymers

Chemical extractability of thebaine, a model opioid drug, as a mixture with a non-gel forming excipient, Avicel pH 102, or selected gel-forming polymers, was tested using absolute ethanol. Of note, the equilibrium solubility of thebaine in ethanol was measured to be 10.4 mg/mL (in 24 h) and 10.5 mg/mL (in 72 h). Physical mixtures of thebaine (600 μg) with 150 mg Avicel or Avicel with selected polymers (80 mg XG, 20 mg XG+30 mg HEC, or 20 mg XG+60 mg CS) mg) were extracted in 1 mL of absolute ethanol for 1 h. Free thebaine without any excipients was also tested for comparison. Around 80% of thebaine was extracted in 1 h from all mixtures, indicating that those polymers had no effect on the extractability of thebaine by ethanol. This result as well as polymers not forming gels in organic solvents indicate that additional strategies are needed to prevent drug abuse by organic solvents.

### 2) Nanoparticle Development

### 2a) NPs Design and Characterization

Thebaine was encapsulated in polytannic acid/iron-stabilized Pluronic F127 NPs by a quasi-emulsion method (QpTA NPs). FIG. 2 shows the schematic of NP formation. In a quasi-emulsion, the aqueous phase containing thebaine and Pluronic F127 creates transient water-in-oil emulsion based on the viscosity of glycerol. The aqueous internal phase was stabilized by the addition of TA and Fe, which generate TA-Fe (III) coordination complex at the water-glycerol interfacial layer, resulting in thebaine-loaded NPs.

The size, morphology and encapsulation efficiency of NPs were characterized by dynamic light scattering (Zetasizer Nano-ZS90), transmission electron microscopy, and reverse phase (RP) high-performance liquid chromatography (HPLC), respectively. HPLC system was equipped with a UV detector set to 285 nm wavelength (1100 series, Agilent Technologies, Palo Alto, CA, USA). An Ascentis C18 analytical column (250 mm 4.6 mm, particle size 5 m) served as the stationary phase (Supelco, St. Louis, MO, USA). At a flow rate of 1 mL/min, an isocratic 30% ACN and 70% water was utilized as a mobile phase. Prior to analysis, samples were dissolved in 0.1 M HCl and filtered through syringe filters (0.2 m pore size).

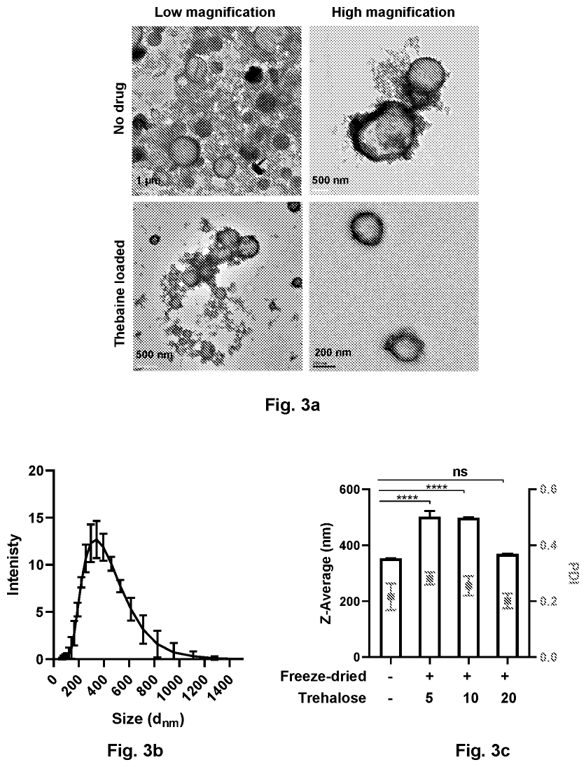

QpTA NPs with or without thebaine were spherical (FIG. 3a) and had an average size of 355.9±17.7 nm with PDI of 0.251±0.018 (FIG. 3b) and a drug loading capacity of ≤10 wt %. QpTAs freeze-dried with trehalose in 20× excess weight were redispersed to the same size as fresh NPs (FIG. 3c).

2b) Chemical Extractability from Nanocapsules.

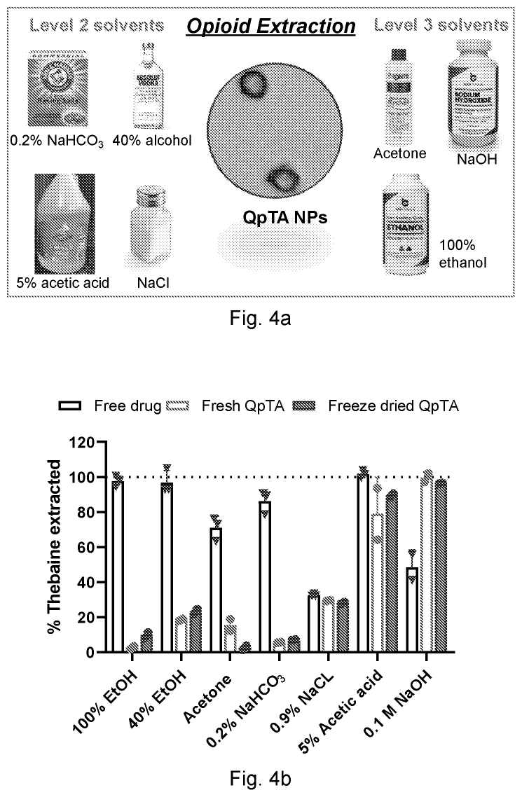

Thebaine as free drug, fresh QpTA NPs, and freeze-dried NPs (1:20 NP: trehalose) was challenged with different household aqueous and organic solvents (or their diluted solutions) including ethanol, one of the most common solvents used by abusers, per the FDA recommendation (FIG. 4a). Thebaine was soluble in all tested solvents except for 0.9% NaCl and 0.1 M NaOH. Fresh or trehalose freeze-dried NPs were dispersed in each solvent and shaken for 1 h, and the drug extracted to the solvent was quantified by HPLC. Of the encapsulated drug, 2.7±1.3% was extracted in absolute ethanol after 1 h incubation at room temperature (FIG. 4b). In the same condition, 5.5±0.2%, 15.5±4.7%, 18.5±0.7% and 29.4±0.42% of the encapsulated drug were extracted in 0.2% NaHCO₃, acetone, 40% ethanol and 0.9% saline, respectively (FIG. 4b). These results indicate that QpTA NPs prevented the extraction of thebaine in absolute ethanol, 40% ethanol, acetone and 0.2% NaHCO₃ unlike unformulated thebaine, which dissolves completely in most solvents at equivalent concentrations. However, NPs did not deter extraction by 5% acetic acid (vinegar) or 0.1 M NaOH, allowing ˜100% of the drug to be extracted in 1 h. This may be due to disassembly of TA-Fe coordination bond or hydrolysis of TA in highly acidic or alkaline pH, respectively. At low pH<2, most of the hydroxyl groups of TA are protonated, leading to immediate destabilization of Fe-TA crosslinking (the stability constant of Fe-TA complex reduced by 4 orders of magnitude from pH 5 to from 2) causing disassembly of the NPs. On the other hand, alkaline pH accelerates TA hydrolysis evidenced by increase of gallic acid in the solution or changes in UV spectra or the diffusion coefficient of TA. There was no significant difference in the extractability between fresh and freeze-dried NPs, which indicates that freeze drying process did not the affect the stability of NPs. These results indicate that QpTAs can add an additional barrier to resist the common household solvents and complement the polymer-based ADF strategies.

### 3. Tablet Production

QpTA NPs were combined with gel-forming polymers to make a tablet by direct compression. NPs experience different forces during tablet processing, such as a drying process, grinding, blending, and compression. The functional stability of QpTA NPs was assessed to test if these forces affect their physical integrity after each processing step by challenging them with ethanol for 1 h. The conditions least detrimental to the stability of QpTA NPs were selected to produce the final NP-Tab.

### 3a) Effect of Drying on QpTAs

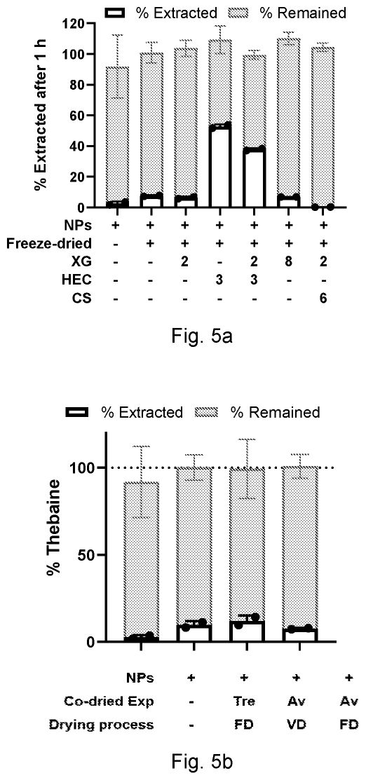

QpTAs collected as an aqueous suspension need to be dried prior to mixing with tablet excipients. QpTAs were initially freeze-dried with trehalose at a weight ratio of 1:20 NPs: trehalose. Ethanol extraction increased no more than by 7% (from 2.7±1.3% of fresh NPs to 9.7±2.2% of freeze-dried NPs) (FIG. 4b). However, NPs dried with trehalose were hygroscopic, making it difficult to homogeneously mix with other excipients. Instead, QpTAs were dried with Avicel pH 102, a tablet binder. QpTAs suspension was mixed with Avicel, and the mixture was freeze-dried for 3 days and subjected to ethanol extraction. The amount of thebaine extracted from Avicel/NP freeze-dried powder was 7.5±0.5% of the total drug, <5% more than fresh NPs (FIG. 5a). NPs co-dried with Avicel in a desiccated vacuum chamber for 24 h showed a similar result (FIG. 5b). These results indicates that QpTA may be dried with a minimal effect on the functional intactness. Avicel/NPs were freeze-dried in the following studies.

### 3b) Effect NPs/Polymers Blending

Avicel/NP powder was blended with ADF polymers such as XG 2% w/v+HEC 3% w/v, XG 2% w/v+CS 6% w/v, or XG 8% w/v, selected from gelation screening in aqueous solutions (FIG. 1). Avicel/QpTA powder was mixed with the polymers and challenged by ethanol to test the compatibility of QpTAs with the polymers (FIG. 5a). It was observed that mixing HEC with XG increased the % drug extraction by ethanol to 37.8±0.9% (FIG. 5a). With each polymer, the % drug extraction was 6.3±0.8% (XG 2%) and 52.8±1.5% (HEC 3%), respectively, which suggests that HEC may cause particle disassembly. HEC may form hydrogen bonding with tannic acid and disrupt the TA-Fe (III) complex to enhance drug release. Based on this finding, HEC was excluded from this study. Avicel/QpTA powder mixed with XG 8% w/v showed 6.9±0.1% of the drug extraction in ethanol. When Avicel/QpTA powder was mixed with XG (2%)+CS (6%), the % drug extraction was negligible, and all the drug remained in the powder, unextracted form. This result indicates that XG and CS were compatible with QpTAs, and their combination could protect the dried NPs from ethanol extraction. Based on these results, XG 8% w/v and XG 2% w/v+CS 6% w/v were used for further studies.

### 3c) Effects of Magnesium Stearate and Compression Pressure

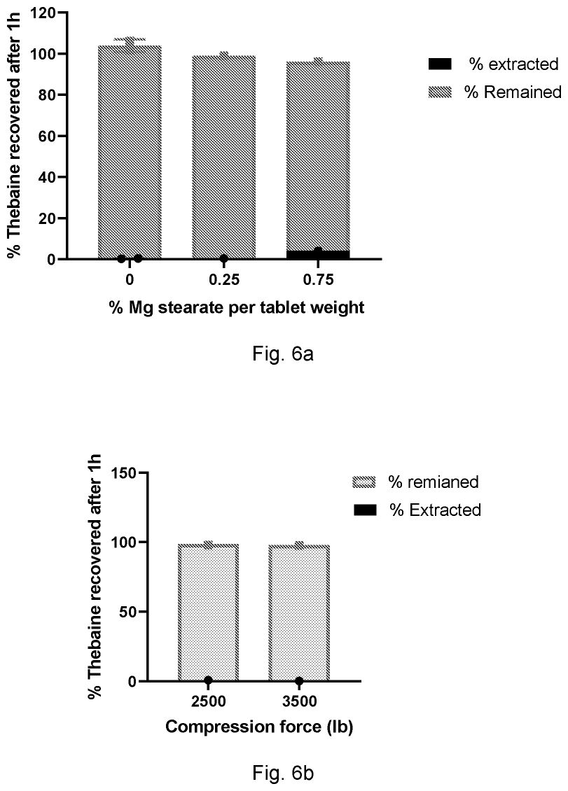

NP-Tab was produced by varying the amount of magnesium stearate and the compression force. Mg stearate at 0.25% w/w of NP-Tab was sufficient to produce a compressed tablet and did not significantly increase the drug extraction by ethanol. However, Mg stearate 0.75% w/w of tablet increased the drug extraction in ethanol from 0.4% to 4% (FIG. 6a). Tablets compressed at 3500 lb resisted drug extraction by ethanol 3.5 times better than those compressed at 2500 lb. Therefore, NP-Tab was produced by compression at 3500 lb for 0.5 min (FIG. 6b).

### 4. Performance of NP-Tab as an Abuse-Deterrent Formulation

A tablet containing thebaine encapsulated in QpTA NPs (NP-Tab) was prepared by the least detrimental conditions to the NP integrity (Table 1). An ADF tablet containing unformulated thebaine (F-Tab) was used as a control. The tablets were subjected to the most common recreational drug users' protocols to assess the effectiveness of abuse-deterrent potential of the NP-Tab against IV abuse. The common attempts used by drug abusers were simulated in the laboratory according to guidelines established by the FDA. Also, we followed new techniques available on various websites and YouTube videos; for example, according to the bluelight blog, acidic liquids such as lemon juice, vinegar, or beer can be used to break the oxycontin tablets apart for easier consumption. Some abusers recommend the usage of sodas or carbonated beverages, or hydrochloric acid to destroy the gelling properties of the current ADF formulations and extract the drug. In all cases, tablets were crushed prior to manipulation for injection.

TABLE 1
Free thebaine ADF tablet (F-tab) and QpTA
encapsulating thebine (NP-tab) composition

| Formulation | F-Tab | NP-Tab |
|---|---|---|
| Drug | ~600 μg, unformulated | ~600 μg, encapsulated |
| | | in QpTA NPs |
| Avicel | 120 mg, physically | 120 mg, freeze-dried with |
| | mixed with drug | QpTA NP suspension |
| ADF polymers | 20 mg XG, 60 mg CS | 20 mg XG, 60 mg CS |
| Magnesium stearate | 0.5 mg | 0.5 mg |
| Compression | 3500 lb, 0.5 min | 3500 lb, 0.5 min |

### 4a) Dispersion in Aqueous Solvents

We evaluated the ability of NP-Tab to resist the standard ways of preparing a drug solution for IV injection. NP-Tab was first pulverized into powder and then hydrated in a small injectable volume of aqueous solvents with different pHs to yield a gel polymer concentration of 8% w/v. The impedance of intravenous abuse was evaluated by (i) the gelation test, showing the ability of NP-Tab powder to form an instantaneous gel, and (ii) syringeability and injectability test, which evaluated the force required to pull and push the obtained gel through a 3 mL syringe equipped with a 21G needle.

### i) Gelation Test:

200 mg NP-Tab was crushed and mixed with 1 mL of each solvent (water, 0.9% NaCl, 0.2% NaHCO₃, 0.1 N NaOH, 5% acetic acid and 0.1 M HCl, 40% ethanol (data not shown) for 1 min. The crushed NP-Tab formed a gel in <5 min in all solvents, confirming the ability of XG+CS to deter drug retrieval in aqueous solvents by gelation. However, the crushed NP-Tab did not form a gel in absolute ethanol and acetone.

### ii) Syringeability and Injectability:

A tension device equipped with load cell of 25 N was used to measure the force required to pull (syringeability) or push (injectability) the NP-Tab powder dispersion, Avicel (bulking excipient) powder dispersion, or water through a 21-Gauge needle. The test method was set up for tension and compression modes for pulling and pushing, respectively. The syringe was pushed or pulled at 1 mm/sec over 20 mm (equivalent to 1 mL). Data was collected by the Wincom software and analyzed using Graphpad prism. Each sample was analyzed 3 times. A dispersion of XG and CS mixture required 5-8 times more force than water or Avicel dispersion.

### 4b) Extraction by Organic Solvents

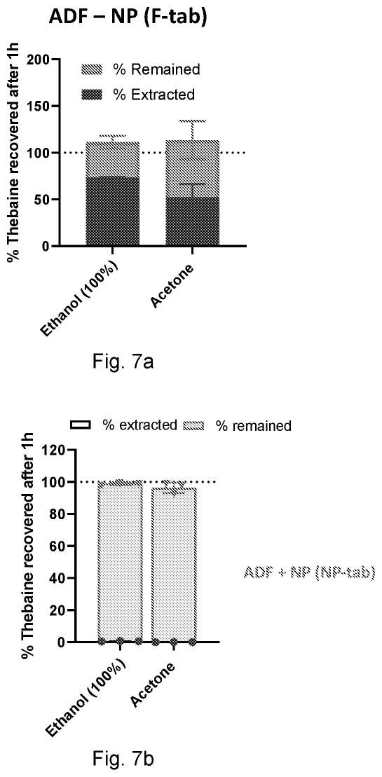

Organic solvents such as ethanol and acetone were used to manipulate the ADFs and extract the drug. The ability of NP-Tab to withstand chemical extractability was investigated. NP-Tabs were crushed, and the powder was incubated with absolute ethanol or acetone at 8% w/v polymer concentration at room temperature for 1 h under shaking. The samples were centrifuged at 16,000 g for 25 min, and the supernatant was filtered and analyzed by HPLC to quantify the extracted drug. The remaining drug was analyzed for mass balance after destroying QpTA NPs in the powder by 0.1M HCl. F-Tab was tested by the same method as a control. Less than 1% of the drug was extracted from NP-Tab, and most of the drug remained in the powder after 1 h in ethanol and acetone (FIG. 7b). From F-Tab, >50% of the drug was retrieved by ethanol or acetone (FIG. 7a). The difference between NP-Tab and F-Tab indicates that Q-pTA NP provides an additional barrier against organic solvent manipulation.

### 4c) Crisping (Thermal Destruction of ADF Polymer)

### i) Laboratory Simulation of Crisping:

The crisping procedure was simulated in the laboratory by a hot plate coupled with a heat block as a heat source. The optimal temperature and time for testing were determined such that they are enough to destroy the polymer (evident from the brown color) and its gelling ability but do not destroy the drug.

First, a mixture of 20 mg XG and 60 mg CS was placed in a glass tube and heated in a heat block at 160-165° C., 165-170° C., 170-175° C., 175-180° C., or 195-200° C. for 2-10 min until they turned into golden brown. The time frame was adjusted according to the temperature. A shorter time was needed at higher temperatures to crisp the polymers. Samples were then hydrated to a polymer concentration of 8% w/v, and the gelling ability was assessed and scored as described above. Upon heating the polymer mixture at a temperature range from 175-180° C. for 10 min or at 200° C. for 3 min, the polymer did not form a gel for at least 1 h (Score 0) (Table 2).

TABLE 2
Gelation of 20 mg XG + 60 mg CS
in 1 mL of water after heating (crisping).

| | Heating | | | |
|---|---|---|---|---|
| Temp | time | Color after | Gelation after | |
| (° C.) | (min) | heating | heating | Score* |
| RT | 0 | Light yellow | Gelled | 5 |
| 160-165 | 10 | light brown | Gelled | 5 |
| 165-170 | 10 | Light Brown powder | Very viscous | 1 |
| 170-175 | 10 | Light Brown powder | Viscous | 1 |
| 175-180 | 10 | Brown | Liquid | 0 |
| 175-180 | 3 | White | Gelled | 5 |
| 195-200 | 2 | Light yellow | Gelled | 5 |
| 200 | 3 | Brown | Liquid | 0 |

*0: did not gel in 1 h; 1: gelled in 30-60 min; 2: gelled in 10-30 min; 3: gelled in 5-10 min; 4: gelled in 1-5 min; and 5: gelled in 1 min.

Next, the effect of crisping on drug stability was investigated. Thermogravimetric analysis (TGA) showed that thebaine began to decompose at around 200° C. and underwent a multi-step decomposition after that. However, 86.9% of the drug withstood heating at 180° C. for 10 min. Unformulated thebaine was physically mixed with XG+CS and heated at 180° C. for 10 min or 200° C. for 3 min. The drug in the crisped mixture was retrieved by 30 min incubation in 0.1 M HCl. Consistent with TGA and drug stability at 180° C., 60.2% of the drug was recovered after crisping at 180° C. for 10 min, and 26.7% after 200° C. for 3 min. The apparently low drug recovery from the drug/XG+CS mixture compared to the drug alone after 10 min heating at 180° C. (60.2% vs. 86.9%) is likely due to the adsorption of the drug to the polymer mixture, which interfered with drug recovery into 0.1 HCl. Overall, 180° C. for 10 min was destructive to XG+CS polymers but not as much to the drug and, thus, was considered suitable for crisping. We chose 180° C. for 10 min for the crisping challenge.

### ii) Crisping Challenge:

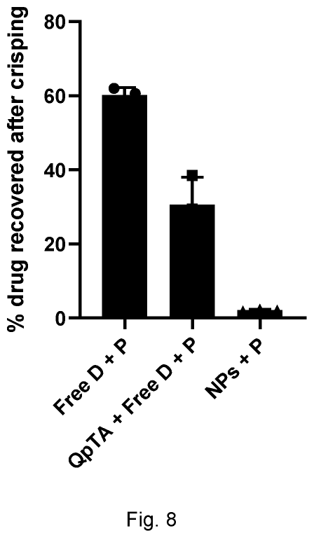

A physical mixture of XG+CS and thebaine-loaded QpTA NPs was heated at 180° C. for 10 min, and the drug was retrieved by 0.1M HCl. After crisping, only 2.1% of thebaine from the polymer and drug-loaded QpTA NP mixture was accessible, whereas 60.2% was recovered from the polymers and unformulated thebaine mixture as shown above (FIG. 8 shows 3 independent batches). This result suggests that QpTA NPs may induce almost complete drug destruction in a condition that >60% of the drug in an unformulated mixture can tolerate. In other words, abusers may not be rewarded by crisping NP-Tab.

To understand how QpTA NPs translate the heat effect to the drug stability, we challenged a mixture of unformulated thebaine, blank QpTA NPs, and XG+CS by crisping. Thebaine in this mixture was destroyed two times more than the thebaine/XG+CS mixture upon crisping, yielding 30.6% drug recovery (FIG. 8). The difference between free drug/polymer mixture (60.2% recovery) and free drug/blank QpTA NP/polymer mixture (30.6% recovery) indicates that QpTA NPs may absorb heat during crisping and transfer the heat to the surrounding drug. However, the fact that drug-loaded QpTA NP/XG+CS mixture (2.1% recovery) lost most drug upon crisping indicates that the intimate contact between drug and QpTA NPs (by encapsulation) was critical to the heat-mediated destruction of drug.

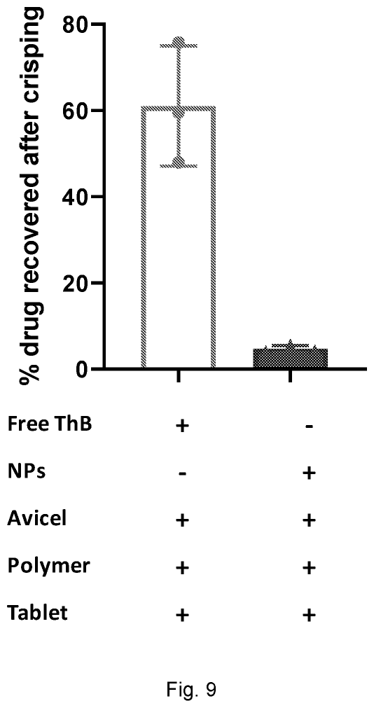

Finally, the effect of crisping was tested on the NP-Tab, F-Tab, and regular tablets (tablets without XG+CS). The tablets were crushed, and powders were heated at 180° C. for 13 min. The heating time was extended by 3 min, because 10 min was not sufficient to break the polymer in tablets. We suspect that 120 mg of Avicel (bulking excipient of a tablet) may have absorbed heat and attenuated the heat effect on the XG+CS polymers. Thirteen min was enough for crisping XG+CS in tablets and did not destroy the drug by more than 40%. The additional extension (to 14- or 15-min heating) caused collateral damage to an unformulated drug and was not suitable for testing the QpTA's contribution to amplifying the heat effect. Consistent with the mixtures tested above, the % recovered from NP-Tab was negligible (4.3% of the total amount of thebaine) after crisping, whereas 60% and 65% thebaine were recovered from F-Tab and regular tablets, respectively (FIG. 9). This result indicates that NP-Tab may deter drug abuse via crisping.

### 5) Dissolution in Simulated Gastric Fluid

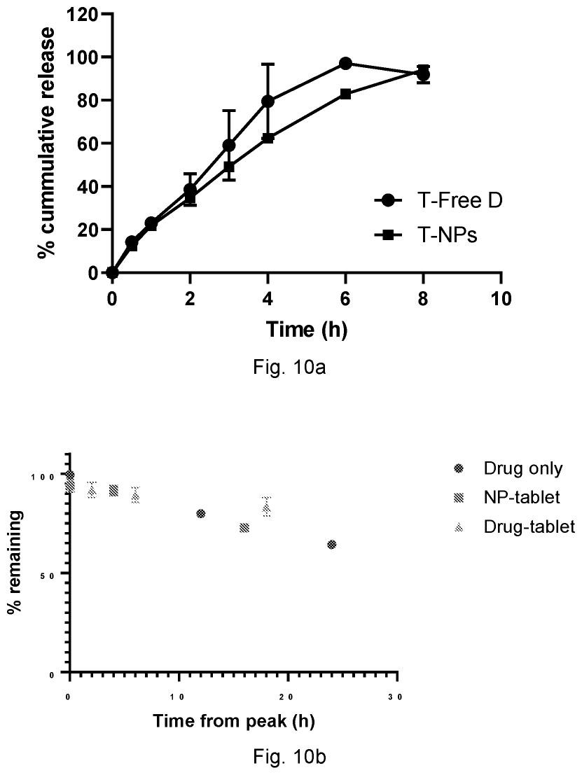

To test the availability of thebaine when the formulation was orally taken as prescribed, in vitro dissolution of the NP-tab was investigated in simulated gastric fluid (SGF). F-Tab was used as a control (Table 1). Tablets were placed in 25 mL of SGF (0.2% NaCl and 0.7% HCl), stirred at 200 rpm at 37° C. At each time point (30 min-24 h), 1 mL of the media was withdrawn and replaced by 1 mL of SGF. Samples were filtered and analyzed using HPLC. The % cumulative release was plotted against time (h). Both NP-Tab and F-Tab showed immediate drug release profiles. Almost 40% of the drug was released from both tablets in 2 h. The NP-Tab showed a relatively slower dissolution profile (6 h) than F-Tab, which dissolved completely within 4 h. Nevertheless, both tablets released almost 100% of drug in 6-8 h (FIG. 10a). Since thebaine degraded in the SGF after 12 and 24 h (FIG. 10b), the release profile was stopped at 8 h. The tablets were less likely to remain in the stomach for more than 6 h depending on the gastric emptying time; therefore, it can be inferred that NP-Tablets can provide the drug and provide the desired analgesic effect if it is taken as recommended orally.

In summary, Tannic acid/iron-stabilized Pluronic NPs complement ADFs. NPs encapsulation can be used in combination with existing ADF strategies to deter drug abuse via IV injection. Tablets consisting of QpTA NPs and gel-forming polymers, such as XG and CS, form a gel instantaneously when crushed and exposed to aqueous solvents with different pHs. The formed gel is very difficult to pull or push through a 21G needle, deterring drug abuse through injection. QpTA NPs do not allow the extraction of the encapsulated drug by organic solvents such as ethanol and acetone, deterring the drug extractability. Moreover, QpTA NPs can deter crisping techniques, which have never been deterred by any marketed products, through their ability to transfer the heat effect to the drug leading to drug degradation, thereby depriving abusers of the drug upon crisping. In conclusion, NP-Tab can develop to deter abusers' techniques to manipulate the tablets for IV injection while retaining its therapeutic effect if taken orally as recommended.

Those skilled in the art will recognize that numerous modifications can be made to the specific implementations described above. The implementations should not be limited to the particular limitations described. Other implementations may be possible. While the inventions have been illustrated and described in detail in the drawings and foregoing description, the same is to be considered illustrative and not restrictive in character, it being understood that only certain embodiments have been shown and described and that all changes and modifications that come within the spirit of the invention are desired to be protected.

It is intended that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

What is claimed is:

1. An abuse-deterrent formulation comprising (i) a drug encapsulated in nanoparticles, wherein the drug is prone to abuse and the nanoparticles are stabilized with polytannic acid alone or in combination with iron, wherein the nanoparticles comprise poloxamer-based surfactants, (ii) a gel-forming polymer, and (iii) one or more pharmaceutically acceptable excipients.

2. The abuse-deterrent formulation of claim 1, wherein the poloxamer-based surfactant is Pluronic.

3. The abuse-deterrent formulation of claim 1, wherein the gel-forming polymer is selected from a starch-based polymer, a polyacrylate, a Carbopol® (CP), a polyethylene oxide (PEO), a cellulose derivative, a polysaccharide, and a combination of two or more polymers.

4. The abuse-deterrent formulation of claim 3, wherein the gel-forming polymer is selected from sodium starch glycolate, sodium polyacrylate starch, Carbopol® (CP), Polyox™ 7 MDa, hydroxyethyl cellulose, xanthan gum and/or chitosan, and a combination of two or more polymers.

5. (canceled)

6. (canceled)

7. (canceled)

8. (canceled)

9. The abuse-deterrent formulation of claim 1, wherein the formulation prevents aqueous or organic solvent extraction, injection, and crisping of the drug.

10. The abuse-deterrent formulation of claim 1, wherein the nanoparticles are resistant to extraction by a common household solvent, wherein the common household solvent is water, ethanol, acetone, an alcohol-containing beverage, vinegar, an electrolyte solution, a sodium bicarbonate solution, or a carbonated beverage comprising phosphoric acid alone or in further combination with caffeine.

11. (canceled)

12. The abuse-deterrent formulation of claim 1, wherein the gel-forming polymer is resistant to extraction by a common household aqueous solvent, wherein the common household aqueous solvent is water, a NaHCO₃ solution, a NaCl solution, a 5% acetic acid solution, a 0.1 M HCl solution, or a 0.1 M NaOH solution.

13. (canceled)

14. The abuse-deterrent formulation of claim 1, wherein the nanoparticles have a diameter range between about 300 nm to about 800 nm, preferably between about 300 nm to about 500 nm.

15. The abuse-deterrent formulation of claim 1, wherein the drug is selected from the group consisting of an opioid, a sedative, a central nervous system (CNS) depressant, a CNS stimulant, and a hallucinogen.

16. The abuse-deterrent formulation of claim 1, wherein the formulation is administered orally in the form of a tablet.

17. The abuse-deterrent formulation of claim 1, wherein the one or more pharmaceutically acceptable excipients are Avicel and magnesium stearate.

18. A method of deterring abuse of a drug, which method comprises providing an abuse-deterrent formulation comprising (i) a drug encapsulated in nanoparticles, wherein the drug is prone to abuse, and the nanoparticles are stabilized with polytannic acid alone or in combination with iron, wherein the nanoparticles comprise poloxamer-based surfactants, (ii) a gel-forming polymer, and (iii) one or more pharmaceutically acceptable excipients, wherein the formulation deters abuse of the drug by preventing an aqueous or organic solvent extraction, injection, and crisping of the formulation.

19. The method of claim 18, wherein the abuse-deterrent formulation is in the form of a tablet, and wherein the drug is encapsulated in an amount of 10 wt % of nanoparticles.

20. (canceled)

21. The method of claim 18, wherein the gel-forming polymer is used in an amount of 60 wt % of 1 g of the tablet.

22. A process for the preparation of an abuse-deterrent formulation comprising:
a) incorporating an abuse-prone drug into nanoparticles;
b) stabilizing the nanoparticles using polytannic acid alone or in combination with iron; and
c) co-formulating nanoparticles with a gel-forming polymer and one or more pharmaceutically acceptable excipients.

23. The process of claim 22, wherein the nanoparticles comprise Pluronic.

24. The process of claim 22, wherein the gel-forming polymer is selected from a starch-based polymer, a polyacrylate, a Carbopol® (CP), a polyethylene oxide (PEO), a cellulose derivative, a polysaccharide, and a combination of two or more polymers.

25. The process of claim 24, wherein the gel-forming polymer is selected from sodium starch glycolate, sodium polyacrylate starch, Carbopol® (CP), Polyox™ 7 MDa, hydroxyethyl cellulose, xanthan gum and/or chitosan, and a combination of two or more polymers.

26. (canceled)

27. The process of claim 22, wherein the step b further comprises lyophilizing the nanoparticles.

28. The process of claim 22, wherein the nanoparticles have a diameter range between about 300 nm to about 800 nm.

29. (canceled)
